(19) United States
(12) Patent Application Publication
(10) Pub. No.: US 2023/0398046 A1
(43) Pub. Date: Dec. 14, 2023

(54) STRUCTURED SURFACTANT BLEND AND SULFATE-FREE AQUEOUS PERSONAL CLEANSING COMPOSITION PREPARED THEREOF

(71) Applicant: RHODIA OPERATIONS, Lyon (FR)

(72) Inventors: Haizhou Zhang, Shanghai (CN); Dawei Jin, Lempeng (SG); Qingrong Song, Shanghai (CN)

(73) Assignee: RHODIA OPERATIONS, Lyon (FR)

(21) Appl. No.: 18/249,321

(22) PCT Filed: Oct. 15, 2020
(86) PCT No.: PCT/CN2020/121068

§ 371 Date: Apr. 17, 2023

(51) Int. Cl. A61K 8/39; A61K 8/49; A61K 8/46; A61K 8/44; A61K 8/92; A61Q 19/10

(52) U.S. Cl. CPC A61K 8/39; A61K 8/4946; A61K 8/466; A61K 8/44; A61K 8/922; A61Q 19/10; A61K 2800/596; A61K 2800/30

(57) ABSTRACT

A structured surfactant blend comprising a first and second anionic surfactant and amphoteric surfactant can form a liquid crystal structure. A sulfate-free aqueous personal cleansing composition comprising the same comprises high level of oil without sacrificing the quality and quantity of foam or lather.

### FIELD OF THE INVENTION

The present invention relates to a structured surfactant blend comprising a first and a second anionic surfactant and an amphoteric surfactant, which can form a liquid crystal structure. The present invention further relates to a sulfate-free aqueous personal cleansing composition comprising the same, which comprise high level of oil without sacrificing the quality and quantity of foam or lather.

### BACKGROUND OF THE INVENTION

At the present time, most of the commercially available personal cleansing compositions are based on sulfate-containing surfactants such as sodium lauryl sulfate (SLS), ammonium lauryl sulfate (ALS), sodium laureth sulfate (SLES) or ammonium laureth sulfate (ALES). SLS, ALS, SLES and ALES are the most widespread sulfate-containing surfactants used in this field as they are inexpensive and as they exhibit at the same time satisfactory cleansing and foaming properties. Another advantage is that they can be easily thickened by the addition of common salt such as sodium chloride.

However personal cleansing compositions including sulfate-containing surfactants present also significant drawbacks. As a matter of fact sulfate-containing surfactants such as SLS are known to be liable to give rise to tolerance problems, especially on the skin and the eyes. Another drawback of sulfate-containing surfactants is their tendency to strip the skin, scalp or hair of its natural oils, fats or proteins contained at their surface. In the long term the repeated use of personal cleansing compositions including sulfate-containing surfactants may therefore cause irritation to the skin or scalp and/or give damage on hair fibers.

Conventional sulfate-free personal cleansing compositions are comprised of water, surfactants, vegetable oils, fragrance, and other minor ingredients such as preservatives, additives, and pigments. Most such sulfate-free personal cleansing compositions are limited to no more than 0.3% fragrance oil and/or vegetable oils which can be incorporated without affecting viscosity, foaming properties, moisturizing properties, deposition on target area. Upon increase of water insoluble compounds such as fragrance oils and vegetable oils with more than 0.5% affects stability, aesthetics and performance in terms of foam yield.

There is a strong interest in this art to provide personal care formulations which tolerate more vegetable oil. One approach to sulfate-free personal care formulations incorporates a surfactant composition consisting of sodium lauroyl methyl isethionate, sodium lauroamphoacetate, sodium cocoyl isethionate, sodium methyl oleoyl taurate, and trisodium ethylenediamine disuccinate (Iselux SLB) available through Innospec, Inc. which is advertised as allowing up to 10% oils or other non-compatible actives.

Structured surfactants, or lamellar liquid crystal-based systems, are becoming very popular to formulate shampoos and body washes that have unique properties and consumer benefits. It is believed that structured surfactants are able to easily emulsify oils with low shear mixing thus allowing high levels of oil in the cleansing formulations without sacrificing the quality and quantity of foam or lather.

Therefore, it is desirable to develop structured surfactant blends so as to prepare personal cleansing formulations which are free of sulfate and incorporate high levels of oil.

### SUMMARY OF THE INVENTION

It is thus one object of the present invention to provide a structured surfactant blend which can form liquid crystal structure thus allowing high levels of oil in the cleansing formulations without sacrificing the quality and quantity of foam or lather.

The structured surfactant blend composition of the present invention comprises
a) a first anionic surfactant represented by formula I having an amount of 15 to 35 wt. %, based on the total weight of the structured surfactant blend composition,

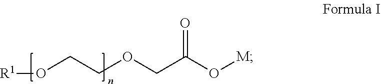

wherein n is an integer from 0 to 30, R¹ is selected from H, alkyl or hydroxylalkyl having 8 to 22 carbon atoms, M is alkali or alkaline earth metal ion;
b) a second anionic surfactant represented by formula II having an amount of 2 to 30 wt. %, based on the total weight of the structured surfactant blend composition,

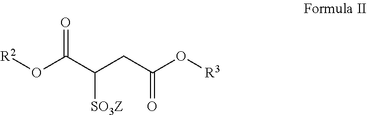

wherein R² and R³ is independently selected from alkyl having 1 to 30 carbon atoms, Z is alkali or alkaline earth metal ion;
c) an amphoteric surfactant selected from alkyl betaine, amidopropyl betaine, sulfobetaine, amphoacetate, amphopropionate, wherein the amphoteric surfactant has an amount of 5 to 35 wt. %, based on the total weight of the structured surfactant blend composition; and
d) a balanced aqueous medium.

In one embodiment of the present invention, the surfactant blend composition further comprises an amino acid based surfactant, preferably the amino acid based surfactant is selected from the surfactants based on glycine, sarcosine, taurine, alanine, aspartic acid, glutamic acid, glutamine and arginine.

In another aspect of the present invention, it is provided a sulfate-free aqueous personal cleansing composition, comprising
i) the structured surfactant blend composition as illustrated above, having an amount of 10 to 70 wt. %, based on the total weight of the sulfate-free aqueous personal cleansing composition;
ii) an oil having an amount of 1 to 50 wt. %, based on the total weight of the sulfate-free aqueous personal cleansing composition.

The aqueous personal cleansing composition of the present invention is free of sulfate, incorporates high levels of oil, and has excellent quality and quantity of the foam or lather.

In yet another aspect of the present invention, it is provided the use of the sulfate-free aqueous personal cleansing composition for washing skin or keratin substrates, in particular the hair or the scalp.

### BRIEF DESCRIPTION OF THE FIGURES

FIG. 1 showed the crystal structure of three surfactant blends under polarization microscope, wherein FIGS. 1a, 1b and 1c showed the photomicrograph of the sample S1, S2 and CS1 respectively.

FIG. 2 showed the foam performance of the four cleansing formulation, wherein FIGS. 2a and 2b showed rich foam, FIGS. 2c and 2d showed minor foam.

### DETAILED DESCRIPTION OF THE INVENTION

Throughout the description, including the claims, the term “comprising one” or “comprising a” should be understood as being synonymous with the term “comprising at least one”, unless otherwise specified. The terms “between” and “from . . . to . . . ” should be understood as being inclusive of the limits.

The articles “a”, “an” and “the” are used to refer to one or to more than one (i.e., to at least one) of the grammatical object of the article.

It should be noted that in specifying any range of concentration, weight ratio or amount, any particular upper concentration, weight ratio or amount can be associated with any particular lower concentration, weight ratio or amount, respectively.

As used herein, the term “alkyl” means a saturated hydrocarbon radical, which may be straight, branched or cyclic, such as, methyl, ethyl, n-propyl, isopropyl, n-butyl, sec-butyl, t-butyl, pentyl, n-hexyl, cyclohexyl.

As used herein, the term “hydroxyalkyl” means an alkyl radical, which is substituted with a hydroxyl groups, such as hydroxymethyl, hydroxyethyl, hydroxypropyl, and hydroxydecyl.

As used herein, the terminology “(Cn-Cm)” in reference to an organic group, wherein n and m are each integers, indicates that the group may contain from n carbon atoms to m carbon atoms per group.

### Structured Surfactant Blend

In one aspect of the present invention, it is provided a structured surfactant blend. The inventors surprisingly found the structured surfactant blend form a liquid crystal structure thus allowing high levels of oil in the cleansing formulations without sacrificing the quality and quantity of foam or lather.

The structured surfactant blend of the present invention comprises,
a) a first anionic surfactant represented by formula I having an amount of 15 to 35 wt. %, based on the total weight of the structured surfactant blend composition,

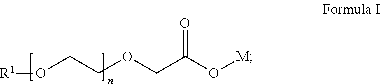

wherein n is an integer from 0 to 30, R¹ is selected from H, alkyl or hydroxylalkyl having 8 to 22 carbon atoms, M is alkali or alkaline earth metal ion;
b) a second anionic surfactant represented by formula II having an amount of 2 to 30 wt. %, based on the total weight of the structured surfactant blend composition,

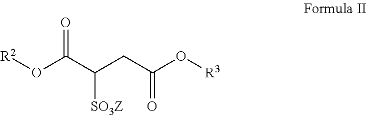

wherein R² and R³ is independently selected from alkyl having 1 to 30 carbon atoms, Z is alkali or alkaline earth metal ion;
c) an amphoteric surfactant selected from alkyl betaine, amidopropyl betaine, sulfobetaine, amphoacetate, amphopropionate, wherein the amphoteric surfactant has an amount of 5 to 35 wt. %, based on the total weight of the structured surfactant blend composition; and
d) a balanced aqueous medium.

In one embodiment of the present invention, the first anionic surfactant has an amount of 16 to 25 wt. %, based on the total weight of the structured surfactant blend composition.

The first anionic surfactant is represented by formula I

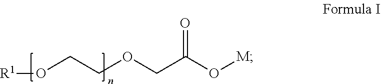

wherein n is an integer from 0 to 30, preferably 0 to 20, more preferably 1 to 15; R¹ is selected from H, alkyl or hydroxylalkyl having 8 to 22 carbon atoms, preferably 8 to 18 carbon atoms, more preferably 11 to 13 carbon atoms; M is alkali or alkaline earth metal ion.

In one embodiment of the present invention, the first anionic surfactant is selected from sodium laureth-13 carboxylate, commercially available from Solvay under the trade name of Miranate® LEC-80, or sodium laureth-4 carboxylate, commercially available from Solvay under the trade name of Miranate® L4C.

In one embodiment of the present invention, the second anionic surfactant represented by formula II has an amount of 4 to 12 wt. %, based on the total weight of the structured surfactant blend composition.

The second anionic surfactant is represented by formula II

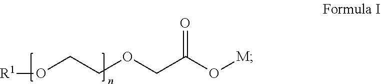

wherein R² and R³ is independently selected from alkyl having 1 to 30 carbon atoms, preferably alkyl having 6 to 12 carbon atoms;
Z is alkali or alkaline earth metal ion.

In one embodiment of the present invention, R² is the same as R³.

In one embodiment of the present invention, R² and R³ are independently selected from alkyl having 8 to 22 carbon atoms.

In one embodiment of the present invention, the second anionic surfactant is selected from diethylhexyl sodium sulfosuccinate, which is commercially available from Solvay under the trade name of Geropon CYA-75P or Aerosol OT 75.

The amphoteric surfactants comprised in the structured surfactant blend composition of the present invention are selected from alkyl betaine, amidopropyl betaine, sulfobetaine, amphoacetate, amphopropionate, wherein the amphoteric surfactant has an amount of 5 to 35 wt. %, preferably 5 to 15 wt. %, based on the total weight of the structured surfactant blend composition.

In one embodiment of the present invention, the surfactant blend composition further comprises an amino acid based surfactant, preferably the amino acid based surfactant is selected from the surfactants based on glycine, sarcosine, taurine, alanine, aspartic acid, glutamic acid, glutamine and arginine.

In another embodiment of the present invention, the amino acid based surfactant is selected from C₈-C₁₆ alkyl glycinate.

The structured surfactant blend of the present invention may further comprise aqueous medium. In one embodiment of the present invention, the aqueous medium is selected from water. In another embodiment of the present invention, the aqueous medium is a mixture of water and organic solvent miscible with water, such as for example, ethanol, propanol, butanol, ethylene glycol, propylene glycol, butylene glycol, glycerin.

In the present invention, the structured surfactant blend composition can be provided as various form according to actual needs, such as solution, paste, suspension and the like.

### Sulfate-Free Aqueous Personal Cleansing Composition

In another aspect of the present invention, it is provided a sulfate-free aqueous personal cleansing composition comprising the structured surfactant blend as illustrated above. Without being bound to any particular theory, it is believed that the structured surfactant blend allows high levels of oil in the sulfate-free aqueous personal cleansing composition comprising the same without sacrificing the quality and quantity of foam or lather, because the structured surfactant blend can form liquid crystal structure.

The sulfate-free aqueous personal cleansing composition of the present invention comprises:
i) the structured surfactant blend composition as illustrated above, having an amount of 10 to 70 wt. %, preferably 30 to 60 wt. %, more preferably 40 to 55 wt. %, based on the total weight of the sulfate-free aqueous personal cleansing composition;
ii) an oil having an amount of 1 to 50 wt. %, preferably 3 to 30 wt. %, more preferably 5 to 10 wt. %, based on the total weight of the sulfate-free aqueous personal cleansing composition.

In the present invention, the oil has different use in different formulations, for example, emollient agent in shower gel or facial cleanser, conditioning agent in shampoo.

In the present invention, the oil is the one or more selected from the group consisting of vegetable oil, fatty acid ester, animal fat, fatty acid and alcohol, silicone oil and paraffin oil.

In the present invention, vegetable oil refers to both hydrogenated and unhydrogenated vegetable oils. Examples of such vegetable include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower oil, hydrogenated safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower oil, and mixtures thereof.

Examples of fatty acid esters include but not limited to butyl myristate, cetyl palmitate, decyloleate, glyceryl laurate, glyceryl ricinoleate, glyceryl stearate, glyceryl isostearate, hexyl laurate, isobutyl palmitate, isocetyl stearate, isopropyl isostearate, isopropyl laurate, isopropyl linoleate, isopropyl myristate, isopropyl palmitate, isopropyl stearate, propylene glycol monolaurate, propylene glycol ricinoleate, propylene glycol stearate, and propylene glycol isostearate,

Examples of the animal fat includes but not limited to acetylated lanolin alcohols, lanolin, lard, mink oil and tallow.

Examples of the fatty acids and alcohols but not limited to behenic acid, palmitic acid, stearic acid, behenyl alcohol, cetyl alcohol, eicosanyl alcohol and isocetyl alcohol.

The composition of the invention may further comprise additional optional ingredients which may bring specific benefits for the intended use. Such optional ingredients may include colorants, pearlescent agents, emollients, hydrating agents, opacifiers, preservatives and pH adjusters. The skilled person is able to select according to general knowledge in the art of formulating personal cleansing compositions such as shampoos, shower gels and liquid hand soaps, and the vast literature there-related, appropriate such optional ingredients for application purposes.

In one embodiment, the composition of the present invention comprises a benefit agent selected from insoluble or partially insoluble ingredients such as moisturizers or conditioners, hair coloring agents, anti-UV agents, anti-wrinkle agents, fragrances or essential oils, skin-coloring agents, anti-dandruff agents, and provides enhanced deposition of such benefit agent on the substrate, ex. Hair and/or skin or fabric or counter top or plant leaves.

The composition of the present invention is used in a manner know in the art, for example, in the case of a cleanser or shampoo, by application of the cleanser or shampoo to the skin and/or hair and optionally rinsing the cleanser or shampoo off of the skin and/or hair with water. In one embodiment of the present invention, the sulfate-free aqueous personal cleansing composition is a shampoo, shower gel, facial cleanser, or makeup remover.

According to any one of the invention embodiments, the composition of the invention may have a pH comprised between 4 and 11.

Personal cleansing compositions are usually prepared by mixing individual surfactants and conditioning agents. These components may be supplied as concentrated solutions which are diluted and/or and combined in appropriate ratios by the formulator. The invention covers any surfactant concentrate to be used as component ingredient to prepare a composition of the invention, and especially to surfactant concentrates containing limited levels of water (more advantageous from a cost and environmental perspective).

### EXAMPLES

Materials:
Miranate L4C: Laureth-4 carboxylate, available from Solvay
Geropon CYA-75P: Diethylhexyl sodium sulfosuccinate, available from Solvay
Mackanate LO: Disodium Lauryl sulfosuccinate, available from Solvay
Miranol Ultra L-32: Sodium Lauroamphoacetate, available from Solvay
Mackstat DM: preservative, available from Solvay
Geropon CG3S: Sodium Cocoyl Glycinate, available from Solvay

### Example 1: Structured Surfactant Blend

The structured surfactant blend (51) were prepared as follows:
1. adding DI water in the main batch and then adding Miranol Ultra L-32, dispersing evenly;
2. preheating Miranate L4C around 60-65° C. in a separate tank until a flowable creamy liquid was obtained;
3. adding the liquid-form Miranate L4C in the main batch, dispersing evenly;
4. gradually charging Geropon CYA-75P in the main batch, and dispersing evenly to provide a translucent paste like blend.

A comparative surfactant blend (sample CS1) was prepared according the above process using Mackanate LO instead of Geropon CYA-75P. In the following tables, “5” refers to samples and “CS” refers to Comparative Samples.

TABLE 1
formulation of the structured surfactant blend

| Component (pbw) | S1 | S2 | CS1 |
|---|---|---|---|
| Miranate L4C | 30 | 23 | 30 |
| Geropon CYA-75P | 6 | 6 | — |
| Mackanate LO | — | — | 6 |
| Miranol Ultra L-32 | 7 | 7 | 7 |
| Geropon CG3S | | 7 | |
| Water | To 100 | To 100 | To 100 |

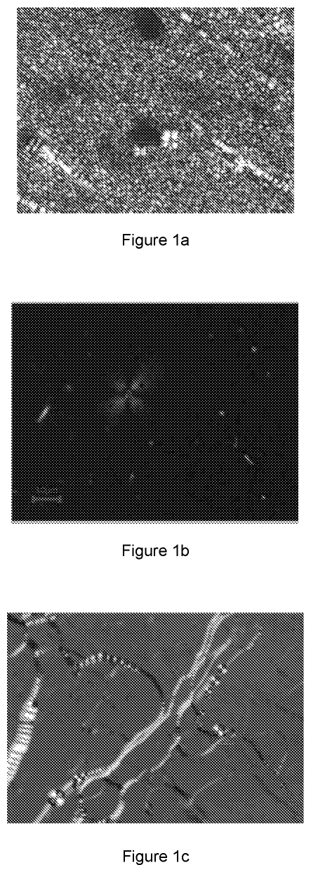

The three samples were observed under polarization microscope (Nikon Eclipse Ti Microscope) at room temperature. FIGS. 1a, 1b and 1c shown the photomicrograph of S1, S2 and CS1 respectively. As shown in FIGS. 1, S1 and S2 shown the liquid crystal structure, while not for CS1.

### Example 2: Sulfate-Free Aqueous Personal Cleansing Composition

The sulfate-free aqueous personal cleansing compositions (sample S3, S4, CS2 and CS3) were prepared as follows:
1. adding DI water into the main pot;
2. adding the surfactant blend (S1 or S2) to the main pot and stirring for 15 minutes at 160 rpm;
3. adding sunflower oil to the main pot and stirring for 5 minutes at 300 rpm, then stirring for further 15 minutes at 160 rpm until the sample was fully emulsified;
4. adding preservative, and stirring for 15 minutes at 160 rpm; and
5. adding citric acid solution (50%) and stirring for 1 hour at 160 rpm.

The Appearance was determined in visual after the formulation was obtained. The viscosity was determined by Brookfield at room temperature.

TABLE 2
formulation of the sulfate-free aqueous
personal cleansing composition

| Component (pbw) | S3 | S4 | CS2 | CS3 |
|---|---|---|---|---|
| Miranate L4C | 15 | 11.5 | 15 | 15 |
| Geropon CYA- | 3 | 3 | | 3 |
| 75P | | | | |
| Mackanate LO | | | 3 | |
| Miranol | 3.5 | 3.5 | 3.5 | |
| Ultra L-32 | | | | |
| Geropon CG3S | | 3.5 | | |
| sunflower oil | 30 | 30 | 30 | 30 |
| Mackstat DM | q.s. | q.s. | q.s. | q.s. |
| Citric acid | q.s. | q.s. | q.s. | q.s. |
| water | to 100 | to 100 | to 100 | to 100 |
| Appearance | Cream- | Cream- | liquid- | liquid- |
| | like, | like, | like, | like, |
| | homo- | homo- | Phase | Phase |
| | genous | genous | separation | separation |
| Viscosity | 30,000 cPs | 20,000 cPs | 1,500 cPs | 2,000 cPs |
| Foam during | very rich | very rich | minor | minor |
| rinsing | foam | foam | foam | foam |

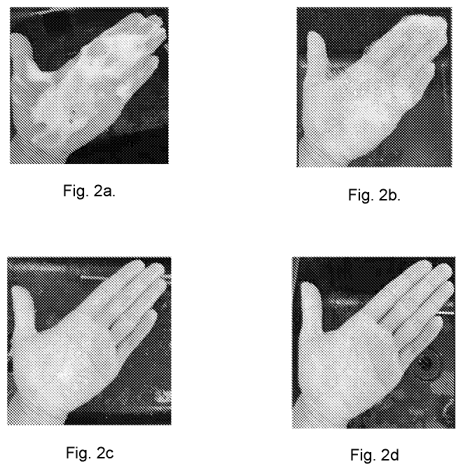

As shown by table 2, the sample S3 and S4 comprised the surfactant blend of the present invention were stable in terms of appearance and viscosity, which proves the sample S3 and S4 tolerate high level of oil. In addition, the sample S3 and S4 have better foam performance than CS2 and CS3 as shown by FIG. 2.

1. A structured surfactant blend composition, comprising,
a) a first anionic surfactant represented by formula I having an amount of 15 to 35 wt. %, based on the total weight of the structured surfactant blend composition, 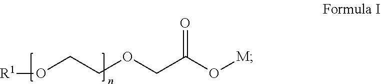
wherein n is an integer from 0 to 30, R¹ is selected from H, alkyl or hydroxylalkyl having 8 to 22 carbon atoms, M is alkali or alkaline earth metal ion;
b) a second anionic surfactant represented by formula II having an amount of 2 to 30 wt. %, based on the total weight of the structured surfactant blend composition, 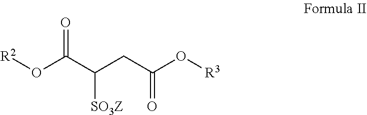
wherein R² and R³ is independently selected from alkyl having 1 to 30 carbon atoms, Z is alkali or alkaline earth metal ion;
c) an amphoteric surfactant selected from alkyl betaine, amidopropyl betaine, sulfobetaine, amphoacetate, amphopropionate, wherein the amphoteric surfactant has an amount of 5 to 35 wt. %, based on the total weight of the structured surfactant blend composition; and
d) a balanced aqueous medium.

2. The surfactant blend composition according to claim 1, wherein R¹ is selected from alkyl or hydroxylalkyl having 8 to 18 carbon atoms.

3. The surfactant blend composition according to claim 2, wherein R¹ is selected from alkyl or hydroxylalkyl having 11 to 13 carbon atoms.

4. The surfactant blend composition according to claim 1, wherein n is an integer of 1 to 20.

5. The surfactant blend composition according to claim 1, wherein R² and R³ and the same.

6. The surfactant blend composition according to claim 1, wherein R² and R³ are independently selected from alkyl having 8 to 22 carbon atoms.

7. The surfactant blend composition according to claim 1, wherein the surfactant blend composition further comprises an amino acid based surfactant.

8. The surfactant blend composition according to claim 7, wherein the amino acid based surfactant is selected from the surfactants based on glycine, sarcosine, taurine, alanine, aspartic acid, glutamic acid, glutamine and arginine.

9. The surfactant blend composition according to claim 7, wherein the amino acid based surfactant is selected from C₈-C₁₆ alkyl glycinate.

10. A sulfate-free aqueous personal cleansing composition, comprising
i) the structured surfactant blend composition according to claim 1 having an amount of 10 to 70 wt. %, based on the total weight of the sulfate-free aqueous personal cleansing composition; and
ii) an oil having an amount of 1 to 50 wt. %, based on the total weight of the sulfate-free aqueous personal cleansing composition.

11. The sulfate-free aqueous personal cleansing composition according to claim 10, wherein the oil is the one or more selected from the group consisting of vegetable oil, fatty acid ester, animal fat, fatty acid and alcohol, silicone oil and paraffin oil.

12. The sulfate-free aqueous personal cleansing composition according to claim 10, wherein the sulfate-free aqueous personal cleansing composition is a skin or hair cleansing composition.

13. The sulfate-free aqueous personal cleansing composition according to claim 10, wherein the composition is used for washing skin or keratin substrates.
